(19) United States
(12) Patent Application Publication
(10) Pub. No.: US 2004/0260360 A1
(43) Pub. Date: Dec. 23, 2004

(54) Skeletal muscle control by means of neuro-electrical signals

(76) Inventors: Eleanor Schuler, Rio Rancho, NM (US); Claude K. Lee, Reno, NV (US)

Correspondence Address: BARNES & THORNBURG, P.O. BOX 2786, CHICAGO, IL, 60690-2786, US

(21) Appl. No.: 10/871,928

(22) Filed: Jun. 18, 2004

Related U.S. Application Data
(60) Provisional application No. 60/479,407, filed on Jun. 18, 2003.

(51) Int. Cl. A61N 1/18

(52) U.S. Cl. 607/48

(57) ABSTRACT

A method and device for skeletal muscle control: The method comprises selecting neuro-electrical coded signals from a storage area that are representative of body organ function. The selected neuro-electrical coded signals are then transmitted to a treatment member, which is in direct contact with the body, and which then broadcasts the neuro-electrical coded signals to a specific body skeletal muscle nerve or organ to modulate the body organ functioning. A control module is provided for transmission to the treatment member. The control module contains the neuro-electrical coded signals which are selected and transmitted to the treatment member, and computer storage can be provided for greater storage capacity and manipulation of the neuro-electrical coded signals.

### RELATED APPLICATIONS

This is the non-provisional filing of application Ser. No. 60/479,407, filed Jun. 18, 2003, entitled “Skeletal Muscle Control by Means of Neuro-Coded Signals.”

### BACKGROUND OF THE INVENTION

This invention relates to a device and method for skeletal muscle control by means of neuro-electrical coded signals.

The principal muscles of the skeleton of humans or animals are operated by the brain. Muscle contraction and movement is coordinated and commanded by many parts of the brain, including the cerebral cortex, cerebellum and brainstem, structures. Instructions in the form of neuro-electrical signals (“signals”) travel to the muscles and cause graduated course or fine motor movements to accomplish the designated task.

Locomotion from place to place via arm, leg and other muscular movements are common to human and all other mammalian life. Building of tools and structures with the muscles of the fingers, hands and arms make the great progress that has historically happened. In the process of steering and directing the actions of muscles over the entire body, the brain accomplishes the duties and desires of everyday life in humans and animals. This invention offers a way to provide more-or-less normal muscular activity by means of the same natural and actual neuro electrical signals that regulate the generally smooth, ever changing symphonic patterns of movement throughout life.

Neuro electrical signals which are sent from the brain to a muscle to command it to contract are carried by the nerves as electrically coded signals. These natural signals vary by the type and assemblage of neuron(s) participating in the signal. There are approximately 300 kinds and types of neurons. A neuron is a cell that has the capacity to generate a repeatable electrical signal. Signal components may vary regarding the voltage, frequency and amplitude change to make the muscle perform its duty.

To operate correctly, muscles first require a source of nutrients and oxygen which are supplied by the blood stream and require intact nerves to carry commands. The neuro commands to a muscle occur in the form of neuro-electrical coded signals via efferent nerves that originate in the central nervous system (brain) and later conduct the signals on the peripheral nervous system. Biological and positional status information is passed from the muscle to the brain by afferent nerves to coordinate and signal that the muscle movement has been completed or is in the process of being completed. Balance and fine motor movements are the final actions and reactions that complete the task(s).

In the un-diseased and uninjured human or animal the movement from place to place, the handling of food, tools, clothing and other implements of daily life occurs effortlessly and as the owner of the muscles and brain desires.

Motor movements are impaired by disease or injury of muscles, or by crushed or severed nerves leading to such muscles. Specific injuries to the brain, spinal cord or skeleton as well as well as lack of oxygen and other nutrients because of cardiovascular failure or damage to the respiratory tract can occur and damage muscle operation.

The ability to cause muscle movement in humans or animals who have spinal or other injuries that make it impossible, difficult or painful to move skeletal muscles would be greatly helped if signals could be controlled by the patient or a care-giver to allow movement via muscular control with natural neuro-electrical coded signals that replace those that are no longer available to operate the affected muscles.

A representative sampling of the muscles which can be controlled and regulated by neuro-electrical coded signals: (The list is not meant to be complete or all encompassing, but to provide a picture of the arena in which the invention operates.)

A. Muscles of the Trunk:

Multifidis; transverse abdominis; rectus abdominis; quadratus lumborum; Iliacus; Internal and external intercostals; Internal and external oblique; rotators and erector spinae.

B. Muscles of the Head, Face and Neck:

Occipitofrontalis; masseter; temporalis; sternocleidomastoideus.

C. Muscles of Hip and Thigh:

Quadriceps; hamstrings; adductors; sartorius; piriformis; hip rotators; gluteus maximus, medius & minimus and pectineus.

D. Muscles of the Shoulder and the Arm to the Elbow:

Levator scapulae; Pectoralae; Deltoides; Biceps; Triceps; subcapularis; Infra and supraspinatae; Rhomboides and Trapezius.

E. Muscles of Hand and Lower Arm:

Finger Digitorus Extensor; Supinator; Finger & Wrist Flexors; Pronator Teres and brachio radialis.

F. Muscles of Foot and Leg:

Flexor digitorm Longus; Flexor Hallucis Longus; Soleus; Fibularis Longus and Brevis; Extensor Digitorum Longus; extensor Hallucis Longus and Tibialis Anterior.

The invention works by recording the actual signals in the nerves leading to each of the skeletal muscles in normal humans or animals.

Patients who suffer from paraplegia or quadriplegia which are largely victims of spinal injuries, can benefit because stored neuro-electrical signals can be transmitted directly into the affected muscles and do not require transmission via the spinal cord. Signals can be coordinated via a small computer to operate one or more muscles to accomplish movement and to complete tasks.

Signals acquired from un-injured humans or animals shall be useful for medical treatment in those who cannot transmit neuro signals from the brain to a muscle due to spinal cord or other injuries. This also applies to individual muscles which need to be exercised by the neuro-electrical coded signals similar to those that previously operated the muscle.

### SUMMARY OF THE INVENTION

The invention provides a method for controlling skeletal muscles. Stored neuro-electrical coded signals that are generated and carried in the body are selected from a storage area. The selected waveforms are then transmitted to a treatment member which is in direct contact with the body. The treatment member then broadcasts the selected neuro-electrical coded signals to a muscle in the body.

The neuro-electrical coded signals may be selected from a storage area in a computer, such as a scientific computer. The process of transmitting the selected neuro-electrical coded signals can either be done remotely or with the treatment member connected to a control module. The transmission may be seismic, electronic, or via any other suitable method.

The invention further provides an apparatus for controlling skeletal muscles. The apparatus includes a source of collected neuro-electrical coded signals that are indicative of skeletal muscle functioning, a treatment member in direct contact with the body, means for transmitting collected waveforms to the treatment member, and means for broadcasting the collected neuro-electrical coded signals from the treatment member to a skeletal muscle.

The transmitting means may include a digital to analog converter. The source of collected waveforms preferably comprises a computer which has the collected waveforms stored in digital format. The computer may include separate storage areas for collected neuro-electrical coded signals of different categories.

The treatment member may be comprised of an antenna or an electrode, or any other means of broadcasting one or more neuro-electrical coded signals directly to the body.

### BRIEF DESCRIPTION OF THE DRAWING

The invention is described in greater detail in the following description of examples embodying the best mode of the invention, taken in conjunction with the drawing figures, in which:

FIG. 1 is a schematic diagram of one form of apparatus for practicing the method according to the invention;

FIG. 2 is a schematic diagram of another form of apparatus for practicing the method according to the invention; and

FIG. 3 is a flow chart of the method according to the invention.

### DESCRIPTION OF EXAMPLES EMBODYING THE BEST MODE OF THE INVENTION

For the purpose of promoting an understanding of the principles of the invention, references will be made to the embodiments illustrated in the drawings. It will, nevertheless, be understood that no limitation of the scope of the invention is thereby intended, such alterations and further modifications in the illustrated device, and such further applications of the principles of the invention illustrated herein being contemplated as would normally occur to the one skilled in the art to which the invention relates.

Natural movement function may require sending electrically reproduced neuro waveforms or coded signals into one or more muscles, including muscles throughout the entire body to control balance locomotion, heavy lifting or the finest motor skills such as threading a needle or writing information or drawing pictures with a pencil, brush or chisel on a stone tablet. This invention utilizes the actual naturally occurring appropriate waveforms or signals to the human or animal muscular array located on the skeleton to activate kinesic, locomotion, fine motor skills, flight, hunting or combat efforts. Such acquired actual and natural signals could be used to adjust or modulate muscle action via conduction or broadcast of neuro-electrical coded signals into such selected nerves as represented by the arms, legs, fingers or foot. The sending or broadcasting of the natural signals may be exerted on the necessary muscles as relates to function(s) required by the central nervous system of humans and animals.

There is or might be some electrical or mechanically induced noise in the signal used for treating muscle disorders. These signals are a close approximation or the exact natural neuro-electrical coded signal but the formation of the signal may have to be electrically filtered, cleaned up, or modified to be more exact in appearance just like the natural codes. But it does represent the actual signal collected from the muscle of a human or animal that is not impaired so as to make it available to a patient who is impaired as to that same signal. Such signals would be the same or closely similar in all mammals or humans.

Skin usually has a 1000 to 30,000 ohm resistance while the interior of the body is quite conductive. All coded signals operate at less than 1 volt, naturally. Applied voltage may be up to 20 volts according to the invention to allow for voltage loss during the transmission or conduction of the required coded signals. Current should always be less than 2 amps output for the invention. Direct conduction into the nerves via electrodes connected directly to such nerves will likely have outputs of less than 3 volts and current of less than one-tenth of an amp. Up to 10 or more channels may be used simultaneously to exert medical treatment on muscular control to aid a patient in moving or performing muscular tasks suitable to his or her well-being as medical treatment.

The invention encompasses both a device and a method for skeletal muscle control by means of neuro-electrical coded signals. One form of a device 10 for skeletal muscle control, as shown in FIG. 1, is comprised of at least one treatment member 12, and a control module 14. The treatment member 12 is in direct contact with a body and receives a neuro-electrical coded signal from the control module 14. The treatment member 12 may be an electrode, antenna, a seismic transducer, or any other suitable form of conduction attachment for broadcasting skeletal muscle signals that regulate or operate muscular function in human or animals. The treatment member 12 may be attached to appropriate nerves, the cervical spine, the neck, or any skeletal muscles in a surgical process. Such surgery may be accomplished with “key-hole” entrance in a thoriac or limb stereo-scope procedure. If necessary a more expansive thoracotomy approach may be required for more proper placement of the treatment member 12. Neuro-electrical coded signals known to modulate skeletal muscle function may then be sent into nerves that are in close proximity with the brain stem.

The control module 14 is comprised of at least one control 16, and an antenna 18. The control 16 allows the device to regulate the signal transmission into the body. As shown in FIG. 1, the control module 14 and treatment member 12 can be entirely separate elements allowing the device 10 to be operated remotely. The control module 14 can be unique, or can be any conventional device which can provide neuro-electrical coded signals for transmission to the treatment member 12.

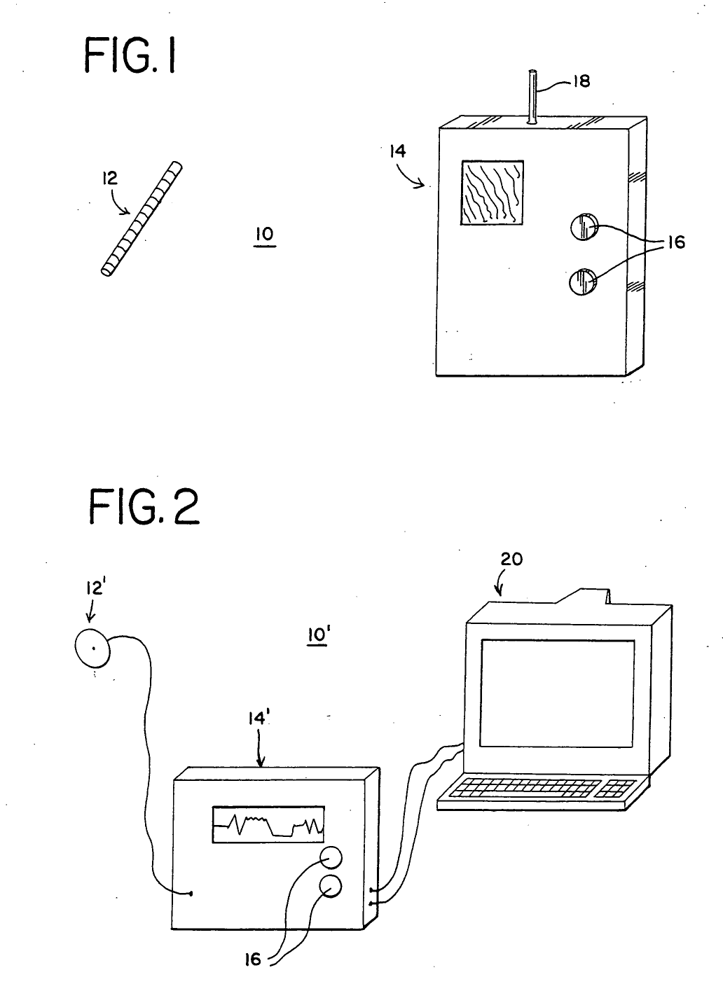

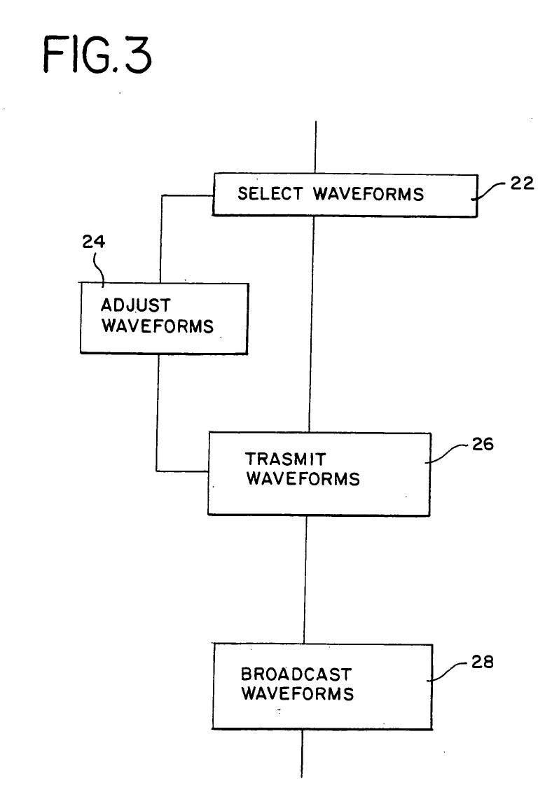

In an alternate embodiment of the device 10, as shown in FIG. 2, the control module 14′ and treatment member 12′ are connected. Similar members retain the same reference numerals in this figure. Additionally, FIG. 2 further shows another embodiment of the device 10′ as being connected to a computer 20, which provides greater capacity to store the neuro-electrical coded signals. The output voltage and amperage provided by the device 10′ during treatment shall not exceed 20 volts nor 2 amps for each signal.

The computer 20 is used to store the unique neuro-electrical coded signals, which are complex and unique to each skeletal muscle and function of the skeletal muscle. It is a neuro-electrical coded signal(s) selected from the stored library of waveforms in the computer 20 which is transmitted to the control module 14′ and used for treatment of a patient. The waveform signals, and their creation, are described in greater detail in U.S. patent application Ser. No. 10/000,005, filed Nov. 20, 2001, and entitled “Device and Method to Record, Store, and Broadcast Specific Brain Waveforms to Modulate Body Organ Functioning,” the disclosure of which is incorporated herein by reference.

The invention further includes a method, as shown in FIG. 3, for using the device 10, 10′ for skeletal muscle control. The method begins at step 22 by selecting one or more stored neuro-electrical coded signals from a menu of cataloged neuro-electrical coded signals. The neuro-electrical coded signals selected activate, deactivate, or adjust the muscular system. Such neuro-electrical coded signals are similar to those naturally produced by the brain structures for balancing and controlling muscular processes. Once selected, the neuro-electrical coded signals may be adjusted, in step 24, to perform a particular function in the body. Alternatively, if it is decided that the neuro-electrical coded signals do not need to be adjusted, step 24 is skipped and the process proceeds directly with step 26. At step 26, the neuro-electrical coded signal is transmitted to the treatment member 12, 12′ of the device 10, 10′.

Upon receipt of the neuro-electrical coded signals, the treatment member 12, 12′ broadcasts the neuro-electrical coded signals to the appropriate skeletal muscle or nerve location, as shown in step 28. The device 10, 10′ utilizes appropriate neuro-electrical coded signals to adjust or modulate muscular action via conduction or broadcast of electrical signals into selected nerves. Controlling skeletal muscle function may require sending neuron electrical coded signals into one or more nerves, including up to ten nerves simultaneously. It is believed that target muscles can only “respond” to their own individual neuro-electrical coded.

In one embodiment of the invention, the process of broadcasting by the treatment member 12, 12′ is accomplished by direct conduction or transmission through unbroken skin in a selected appropriate zone on the neck, head, limb(s), spine, or thorax. Such zone will approximate a position close to the nerve or nerve plexus onto which the signal is to be imposed. The treatment member 12, 12′ is brought into contact with the skin in a selected target area that allows for the transport of the signal to the target nerve(s).

In an alternate embodiment of the invention, the process of broadcasting the neuro-electrical coded signal is accomplished by direct conduction via attachment of an electrode to the receiving nerve or nerve plexus. This requires a surgical intervention as required to physically attach the electrode to the selected target nerve. Direct implantation on the nervous system of the selected muscle or muscle ganglion may be performed in order to transmit signals to control all or some muscle function. Such implantation can be presynaptic or post synaptic and may be attached to ganglion or muscle plexis associated with the desired movement function.

In yet another embodiment of the invention, the process of broadcasting is accomplished by transposing the neuro-electrical coded signal into a seismic form where it is sent into a region of the head, neck, limb(s), spine, or thorax in a manner that allows the appropriate “nerve” to receive and to obey the coded instructions of such seismic signal. The treatment member 12, 12′ is pressed against the unbroken skin surface using an electrode conductive gel or paste medium to aid conductivity.

Various features of the invention have been particularly shown and described in connection with the illustrated embodiments of the invention. However, it must be understood that these particular products, and their method of manufacture, do not limit but merely illustrate, and that the invention is to be given its fullest interpretation within the terms of the appended claims.

We claim:

1. A method for controlling skeletal muscles comprising the steps of:
a. selecting from a storage area one or more waveforms generated in the body and carried by neurons in the body;
b. transmitting or conducting the selected waveforms to a treatment member in contact with the body; and
c. broadcasting the selected waveforms from the treatment member to a muscle in the body is affected to control skeletal muscles.

2. The method according to claim 1, in which step “a” further includes selecting said waveforms from a storage area in a computer.

3. The method according to claim 1, in which step “b” further comprises transmitting the selected waveforms remotely to the treatment member.

4. The method according to claim 1, in which step “b” further comprises seismic transmission of the selected waveforms.

5. An apparatus for controlling skeletal muscles, comprising:
a. a source of collected waveforms generated in the body and indicative of skeletal muscle functioning;
b. a treatment member adapted to be in direct contact with the body;
c. means for transmitting one or more of the collected waveforms to the treatment member; and
d. means for broadcasting the collected waveforms from the treatment member to an area in the body such that a skeletal muscle is affected, thereby controlling skeletal muscles.

6. The apparatus according to claim 5, in which said transmitting means includes a digital to analog converter.

7. The apparatus according to claim 5, in which said source comprises a computer having collected waveforms stored in digital format.

8. The apparatus according to claim 7, in which said computer includes separate storage areas for collecting waveforms of different skeletal muscle functional categories.

9. The apparatus according to claim 5, in which the treatment member comprises an antenna for broadcasting skeletal muscle signals.

10. The apparatus according to claim 5, in which the treatment member comprises an electrode.
